(19) United States
(12) Patent Application Publication
(10) Pub. No.: US 2007/0015990 A1
(43) Pub. Date: Jan. 18, 2007

(54) Method and apparatus for detecting and analyzing pathogenic zones

(76) Inventors: Mark Krinker, Bronx, NY (US); Larry Pismenny, Fair Lawn, NJ (US)

Correspondence Address: Mark Krinker, 140 Alcott Pl., Apt.5-F, Bronx, NY, 10475, US

(21) Appl. No.: 11/169,871

(22) Filed: Jun. 30, 2005

(51) Int. Cl. A61B 5/05

(52) U.S. Cl. 600/407

(57) ABSTRACT

Method and Apparatus for Detecting and Analyzing Pathogenic Zones relate to geophysics and environmental medicine, specifically to searching inauspicious (pathogenic) places. The Method is based on two major concepts. First of them considers influence of extremely low frequency (ELF) electromagnetic (EM) fields, generated by such zones, on human brain and other organs. The danger is based on a spectral compatibility of said external EM fields and those generated by the organs, which affects their pace. The examples are shown. Second concept considers a spinning electric vector (SV). It is formed due to “splitting” Earth alternating electric vector for two vectors, shifted in a phase and spatially. The spinning influences space-time metric, causing the profound consequences for humans. Natural sources of SV are shown. The apparatus comprises a sensor to percept components of Earth electric field. The sensor is connected to ELF-filter and phase-shift meter, which are connected to output indicators.

### BACKGROUND—FIELD OF INVENTION

This invention relates generally both to geophysics and environmental medicine, and more particularly this invention relates to searching auspicious and inauspicious (pathogenic), for a human being, places.

### BACKGROUND—DESCRIPTION OF PRIOR ART

There are descriptions of methods and instruments for detecting inauspicious places, shown in the literature and the patents.

All the methods relate such the places to the distortions of geophysical fields like Earth own electromagnetic fields and others.

The patents of interest are (Ludger Mersmann and others) [L. Mersmann Geo-Magnetometer BPM 2010 www.bio-physica.com, Wassenach, Germany.

A thorough description of the prior art in the field to which this invention pertains may be found in Patent of Youry Kravchenko, Russia, Youry Kravchenko., Indicator of Geophysical Anomaly, Russian Patent n. 2080605, Int. Cl. A 61 N 1/16, May 27, 1997.

It describes a measurement instrument, which measures an integral of a phase shift of Earth own electromagnetic field. The measurement is accomplished within 5-15 kHz band where this field is supported by a natural ground-ionosphere resonator.

When employing the instrument, a reading of the integral of the phase of electric component of said electromagnetic field in a given place is conditionally taken for a zero by an operator. Technically, it's achieved by means of zeroing the reading with the instrument. Then the operator moves to other place and reads the instrument again. If the reading of an integrator exceeds a certain value, then this place is considered to be inauspicious. Said the phase shift can be caused by various reasons like presence subterranean waters, shift of tectonic plates and others.

The prior art suffers from a number of disadvantages.
(a) The phase shift of monochromatic electromagnetic wave, used in the art, is the only measurement parameter by which one considers if the checked area is pathogenic or not.

Another important characteristics, field strength of this wave and its polarization, aren't measured here, what narrows operational capability of the prior art method and the instrument.
(b) The art operates in a narrow band of very low frequency (VLF) portion of electromagnetic spectrum, what gives no attention to other important, human-related portion of the spectrum, like extremely low frequency (ELF) band. The existing art does not register its measured parameter at frequencies, related to human organs. At the present time, the frequencies, related to functioning major human organ and a whole organism, are known to science. Disturbing energetic balance, caused by unwanted electromagnetic spectrum, produced by pathogenic zones, inflicts harm to a human organism. Lack of feature to measure radiations and fields of said ELF-band, narrows an operational capability of the prior art.
(c) The art does not consider a spectral compatibility of the spectrum of its operation and that of human-organs-related frequencies. The lack of said compatibility drastically reduces applications of the art.
(d) The art does not consider spatial phenomena caused by interaction of components of EM wave, related to a landscape or other geophysical characteristics. Spatial phenomena have profound impact on living beings due to fundamental basics of the matter they influence.
(e) The art apparatus registers, beside the useful signal, also unwanted one, caused by two sources: electric pulses, generated by operator's muscles, and the alternative electric field produced due to modulating earth own permanent field by moving limbs of operator's body.
(f) The art can't measure Earth own permanent field, which is an important component in establishing energetic balance between human being and the world.
(g) Limited social application of prior art can be also considered as a drawback: the art detects pathogenic zones, but it can find a human body buried under ruins but by 10 ft deep only.

There is a need, therefore, for the method and the instrument for electronic dowsing which use whole set of characteristics of electromagnetic waves, as well as interaction of EM waves, at frequencies of human organs, as criteria of pathogenic zones, as well as extending its social functions due to providing search of human body, buried under ruins.

Objects and Advantages

The longstanding but heretofore unfulfilled need for the method and the instrument for detecting pathogenic zones on a base of registering a set of EM characteristics at human body-related frequencies are now fulfilled by the invention disclosed hereinafter and summarized as follows.

Accordingly, several objects and advantages of the present invention are:
(a) to provide measurement of extended set of characteristics of earth EM field: phase shift, field strength and polarization for extending received dowsing-related information in VLF- and other bands;
(b) to provide operation of the method and instrument both in ELF and VLF bands related to functioning human organs and the organism as whole.
(c) to provide a spectral compatibility of the method and instrument and that of human-organs-related frequencies, which extends its application.
(d) to provide registration of spatial phenomena caused by interaction of components of EM wave, related to a landscape or other geophysical characteristics.
(e) to make measurement more sensitive due to eliminating unwanted sources of signal, caused both contracting muscles and moving limbs of operator;
(f) to provide measuring Earth own permanent field;
(g) to enlarge a field of social application of method and apparatus due to enabling search of human body, buried under ruins deeper and more precisely.

### DRAWING FIGURES

FIG. 1 shows a generalized unit diagram of the apparatus when interacting with earth

FIG. 2 shows a sensor in details

FIG. 3 shows an ELF-field strength monitoring compartment of the apparatus

FIG. 4 shows a spinning vector compartment of the apparatus

FIG. 5 shows sources of pathogenic zones

FIG. 6 shows origination of the spinning vector

FIG. 7 shows employing the method and instrument for searching human bodies under ruins

### REFERENCE NUMERALS IN DRAWINGS

| | |
|---|---|
| 10 ground | 11 Earth electromagnetic field |
| 12 sensor | 13 output of sensor |
| 14 output of sensor | 15 output of sensor |
| 16 voltage follower | 17 voltage follower |
| 18 voltage follower | |
| 20 ELF processor | 21 ELF field strength indicator |
| 30 spinning vector | 31 spinning vector indicator |
| processor | |
| 40 body of sensor | 41 conductive plate |
| 42 conductive plate | 43 conductive plate |
| 50 shielded body of | 51 ELF filter |
| ELF-compartment | |
| 52 ELF filter | 53 ELF filter |
| 54 ELF vector adder | 51 electrode |
| 60 shielded body of the | 61 band-pass filter |
| unit 30 | |
| 62 band-pass filter | 63 band-pass filter |
| 64 phase shift meter | 65 phase shift meter |
| 66 phase shift meter | 67 vector multiplier with its amplitude inputs |
| | 67 a,b and a phase shift input 67c |
| 68 vector multiplier with | 69 vector multiplier with its amplitude inputs |
| its amplitude inputs | 69 a,b and a phase shift input 69c |
| 68 a,b and a phase shift | |
| input 68c | |
| 70 spinning vector adder | |
| 80 pathogenic zone | 81 liquid lens |
| 82 turbulent | 83 geological heterogeneities |
| subterranean water | |
| 84 Earth own | |
| permanent field | |
| 90 human body | 91 ruins |
| 92 mechanical vibrator | 93 probing waves |
| 94 apparatus | |

Description

FIF. 1 shows the apparatus to find geo-pathogenic (inauspicious) zones of area 10. The apparatus percepts electromagnetic field 11 of this area by means of a sensor 12.

The sensor 12 has tree output channels 13, 14 and 15, which transmit signals of three major mutually-orthogonal component of the field 11. The outputs enter three identical voltage followers 16, 17 and 18. Outputs of the voltage followers 16-18 enter the ELF field strength unit 20 and the spinning vector unit 30. Units 20 and 30 have their output indicators 21 and 31 respectively.

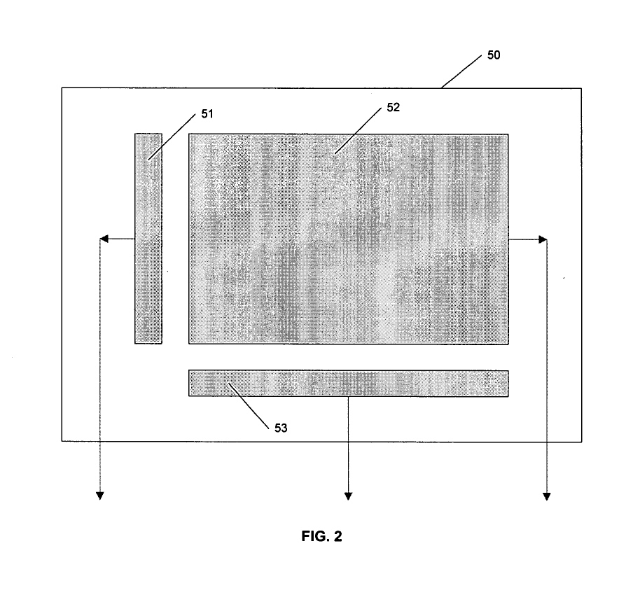

FIG. 2 shows the sensor 12 in details. The sensor has a body 40. There are three mutually—orthogonal conductive plates 41, 42 and 43 inside the body 40. The plates percept signals of major components of electromagnetic field 11. The outputs of the plates form said channels 13, 14 and 15.

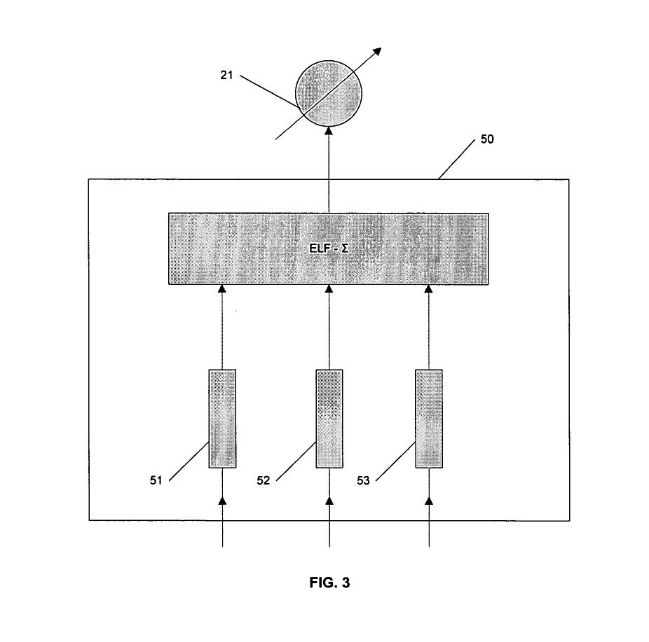

FIG. 3 shows ELF field strength monitoring compartment. It has a shielded body 50 with three identical ELF-filters, 51, 52 and 53, inside. Inputs of said filters receive signals from the outputs of the voltage followers 16-18. Outputs of the ELF filters 51-53 are connected to inputs of the ELF vector adder 54. The output of the adder 54 is connected to the output indicator 21.

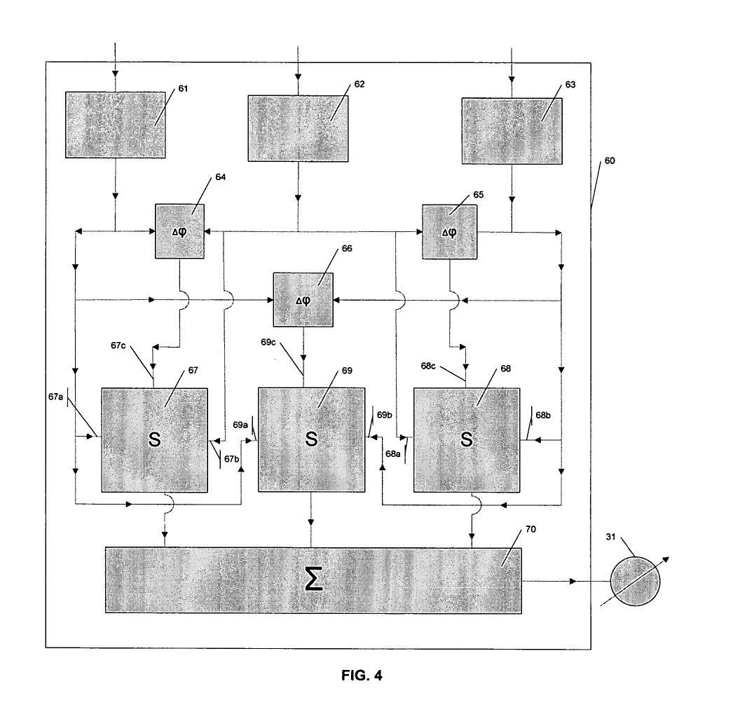

FIG. 4 shows the spinning vector unit 30 in details. The unit 30 has a shielded body 60 with three band-pass filters, 61, 62 and 63, inside. The inputs of said band-pass filters are connected to the outputs of the voltage followers 16-18.

The outputs of the band-pass filters 61-63 are connected to inputs of phase-shift meters 64, 65 and 66. The outputs of the band-pass filters 61, 62 and 63 are also connected to amplitude inputs 67a, b, 68a, b and 69a, b of vector multipliers 67, 68 and 69 correspondingly. The vector multipliers 67, 68 and 69 also have phase shift inputs 67c, 68c and 69c, which are connected to outputs of the phase-shift meters 64, 65 and 66.

The outputs of the vector multipliers 67-69 are connected to the input of the vector adder 70. The output of the vector adder 70 is connected to the meter 31.

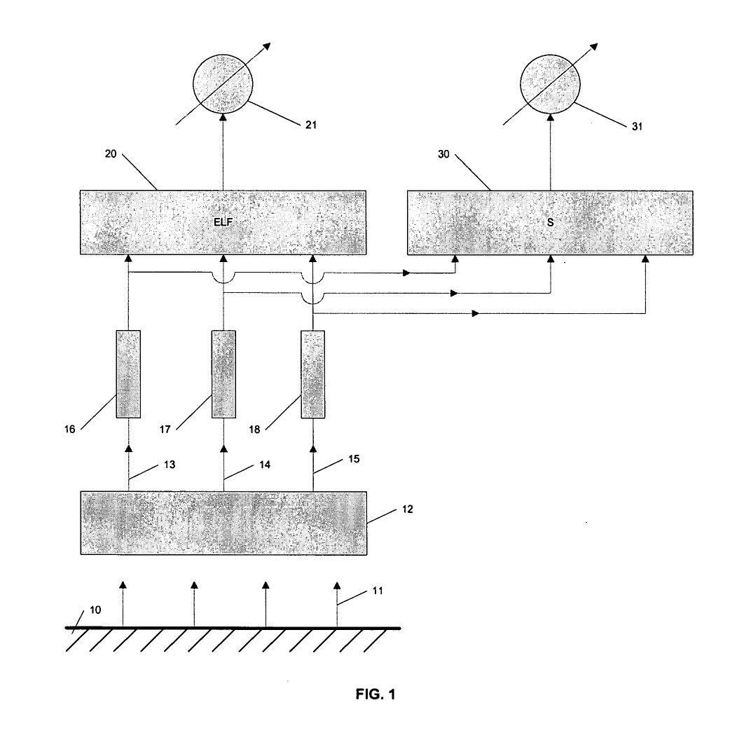

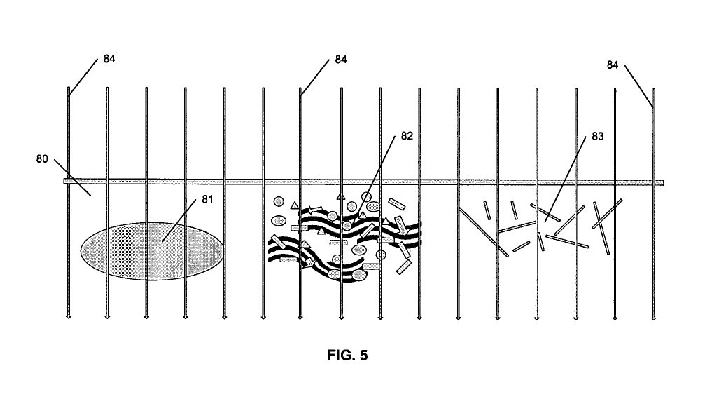

FIG. 5 illustrates possible sources of pathogenic zones. The units, recited above, form a system with a zone 80 (also marked as 10 in the generalized illustration FIG. 1), which includes a subterranean liquid lens 81, subterranean turbulent water 82 and geological heterogeneities 83. All of these are exposed to Earth own electromagnetic field 84 (shown as 11 in FIG. 1). The Earth own field 84 comprises both permanent and alternating components.

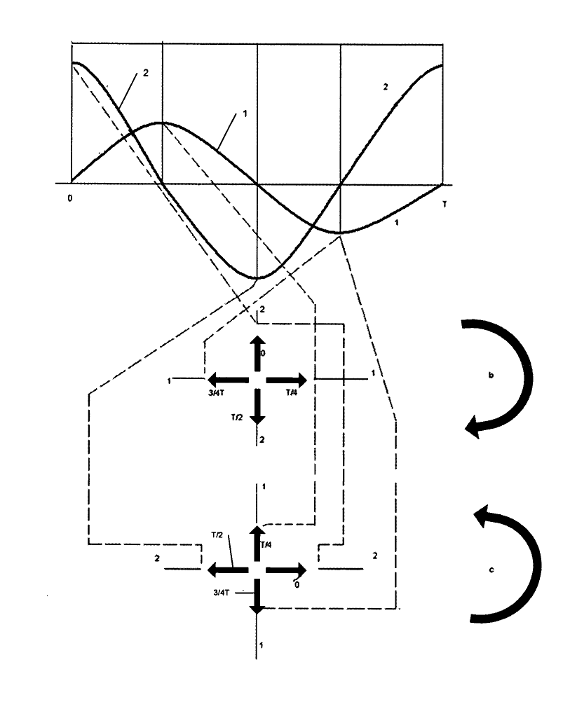

FIG. 6 illustrates origination of a spinning vector, produced by two sine waves 1 and 2 (FIG. 6a), shifted for 90 degrees in a phase, being mutually orthogonal in a space. Their orthogonal space shift is shown with two orthogonal axes 1-1 and 2-2. FIGS. 6b and 6c are different in that the waves 1 and 2 are being swapped spatially. The curved arrows at the right show that direction of the spinning of the resulting spinning vector changes for the opposite when said takes place.

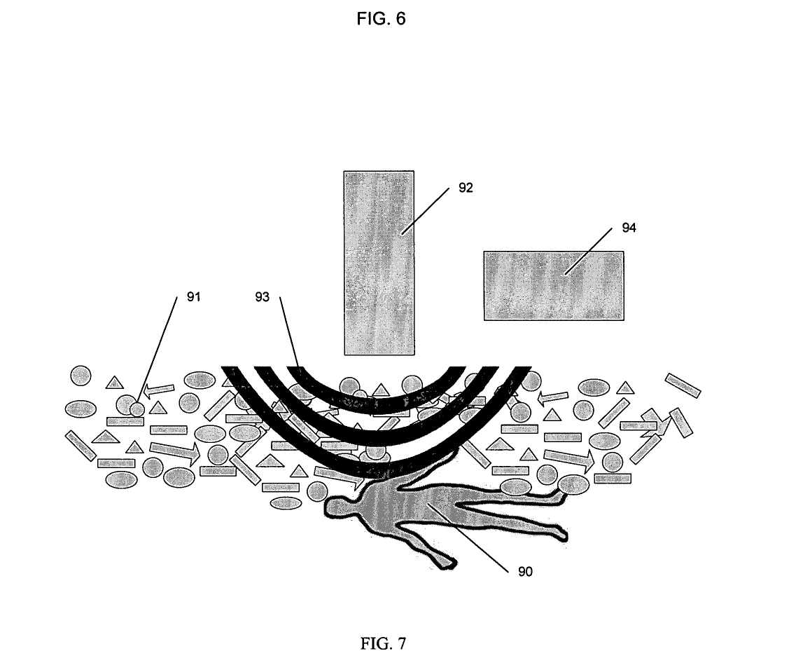

FIG. 7 illustrates conditions for searching human body 90, buried under ruins 91, with employing the mechanical vibrator 92, which produces probing mechanical waves 93, while apparatus 94 percepts electromagnetic fields generated by the body 90 in response to the mechanical waves 93.

Operation

The method and the apparatus work in the following way.

The apparatus is being located over the zone 80 (FIG. 5). The sensor 12 percepts electric components of the field 84 by means of the plates 41-43. All the shown heterogeneities in the ground 80 influence earth own field 84. The liquid lens 81, which can be filled with water, causes additional bound charges when exposed to the field 84. Beside that, this water can cause a phase shift of VLF Earth radiation (with its maximum at 6 kHz) supported by the ground-ionosphere resonator. The additional bound charges increase field strength. The turbulent subterranean water 82 modulates the permanent component of Earth own field due to polarization processes in the water. Due to oscillating water mass, its polarization by Earth permanent field changes according to motion of the water. As a result, an additional AC field origins.

Water-stimulated AC field, or any other ELF field, acts upon the sensor 12, inducing oscillating electric potentials on the plate electrodes 41-43. The oscillating voltages of the electrodes 41-43 enter the voltage followers 16-18, which pass the signals to the ELF-filters 51-53 of the ELF unit 20 and the band-pass filters 61-63 of the spinning vector unit 30. The ELF-filters extract ELF component of the signal caused by additional field, formed due to oscillating turbulent water and other factors.

The ELF portion of the method is based on that the embodied apparatus register electromagnetic fields and waves, which match the frequency band of the human brain and some organs. For the brain it is approximately 1.0-30.0 Hz. If, by any reasons, heterogeneities 81-83 in the ground 80 produce compatible filed, as this was shown above, it will influence the brain. The area of the ground 80 has to be considered as an inauspicious (geopathogenic) in such a case.

As our experiments have shown, the spectrum of originated AC field does match that of electric activity of human brain and other organs in ELF band. The strength of the ELF field, stimulated by turbulent-water, drastically exceeds ELF field of the brain. Turbulent subterranean water can cause 100%-modulation in some cases. That is, if Earth own permanent field is ˜100 V/m, newly originated ELF field reaches the same level. Even being partially shielded with skull bones and tissue, such an intensive field induces electric potential as much as thousands millivolts under a cranium. Human brain produces its own oscillating potentials as much as tens of millivolts, if measured under the cranium. The induced potentials drastically exceed the own ones, causing disturbance of normal brain functioning. Same mechanism refers to human heart and other organs, which are subject to unwanted field, disturbing their pace.

The frequencies of human organs are known today. For specific organs, an ELF band-pass filter with a compatible central frequency can be employed instead the ELF-pass filter.

When measuring Earth permanent field is needed, it can be transformed into alternating one by means of mechanical modulators or electronically. It can be, for instance, a disc with tooth-like openings. When the disc starts to rotate, or the rod starts to oscillate, the earth permanent field converts into the alternating one. Its frequency depends on an angular frequency of the disc and has to be compatible with ELF-pass filter. Then, the originated ELF signal is measured by the apparatus. Its magnitude is proportional to the strength of the Earth permanent field.

Description of the Spinning Vector Portion of the Instrument and the Related Method.

The spinning vector compartment 30 works in the following way. Any of heterogeneities in the ground 80 distorts alternating component of earth own field 84. The water lens 81 creates both refraction of electric vector 84 and the phase shift of its alternating component. The turbulent subterranean water 82 does the similar. The geological heterogeneity 83 refracts the vector 84 and creates its phase-shifted component on joints of the heterogeneities.

As a result, all of the recited subjects produce at least two mutually orthogonal electric vectors (parent-vectors), shifted in a phase. Two mutually orthogonal vectors, shifted in a phase, create the third vector, which spins in a space with a frequency of its parent-vectors.

The sensor 12 has inside its body 40 three mutually orthogonal electrodes 41, 42 and 43. Here, each of the electrodes percepts a corresponding component of external electric vector. Signals of the electrodes enter, through the voltage followers 16-18, band-pass filters 61, 62 and 63. Central frequency of the filters matches that of maximum of the energy, supported by the ground-bottom-of-ionosphere resonator.

In the embodiment of the invention, this frequency was picking 6208 Hz, basing on the research of Earth electromagnetic spectrum. The output signals of the band-pass filters 61-63 are monochromatic sinus waves of voltage or currents. In this embodiment, their frequency is 6208 Hz.

The signals from the outputs of the band-pass filters enter both the phase shift meters 64-66 and the amplitude inputs 67a, b-69a, b of the vector multipliers 67-69.

The phase shift meters 64-66 produce an output voltage (it also can be a current), which is proportional to the phase shift between the input signals. Said phase shift is that between VLF electric vectors allocated along X-, Y- and Z-axis.

Being shifted both spatially and in a phase, each the pairs of these vectors (parent vectors) creates a third vectors, which spatially spins with a frequency of the parent vectors. The spinning of the third vector is shown, in turn, as the vector S, which is normal to a plane of the spinning. Its analytical expression is
S=ωΔφ[E₁×E₂],
where ω,Δφ, and [E_{1,2}] are the angular frequency, phase shift and the vector product of the parent vectors 1 and 2 correspondingly.

For one pair of the parent vectors, the FIG. 6 illustrates the process. FIG. 6-a shows two sine waves, sifted in phase for 90 degrees. The phase-shifted vectors are also shifted spatially. FIG. 6-b and FIG. 6-c show this with two mutually orthogonal axes, on which the sine waves are projected for each of the quarter of the period. The difference in FIG. 6-b and FIG. 6-c is in that the directions 1-1 and 2-2 of the parent vectors are opposite. The spinning of the resulted vector is shown in the right portion of the drawing by means of the curved arrows. As seen, changing spatial orientation of the parent vectors results in changing direction of the spinning of the resulted vector.

The signals at the outputs of the vector multipliers 67-69 represent shown above spinning S for the planes XOY, XOZ and YOZ. The final resulted vector is a sum of these three. Said sum is obtained due to adding the output signals of the vector multipliers 67-69 in the vector adder 70. The indicator 31, connected to the output of the vector adder 70, reads the spinning S.

Appearance of the spinning vector causes a sequence of related events. According to a paradigm of the modern physics, the spinning motion has specific properties in that it produces the Torsion Fields, which change space-time metric with all the following consequences. Human organism is being formed under conditions of the energetic balances with the environment. Any deep distortion especially that of fundamental space-time nature, is harmful to the organism. This is why the appearance of the spinning vector has a profound impact on a human being, and the areas of it localization have to be considered as inauspicious, pathogenic.

According to the theory, developed by Russian prominent astrophysicist N.A. Kozyrev, time and rotation are closely interconnected.

N.A. Kozyrev detected that the weight of the spinning gyroscope changes slightly depending on the angular velocity and the direction of rotation. Reported observations of gyroscope weight variations have been made repeatedly by various researchers in many countries:
Polyakov S. M., Polyakov O. S. “Vvedeniye v eksperimentalnuyu gravitoniku.”, Moscow, Prometei, 1988, 136 p. (Russian) (“Introduction to the experimental gravitonics.”)
Hayasaka H., Takeuchi S. “Anomalous weight reduction on a gyroscope's right rotation around the vertical axis of the Earth.” //Phys.rev.lett., 1989, # 63, p. 2701-2704.
Laithwaite E. R. “The continuing story of gyroscopic magic.” //Elec.rev., 1975, # 197, p. 675-678.

Almost in all cases the observed effects were interpreted as the manifestation of antigravitation that is fundamental changes of space-time take place here. The spinning vector can be considered as a gyroscope. It has a certain mass and an angular frequency. This means that phenomenon of the spinning vector can be related to basics of matter with all the related consequences for human beings.

The method and the apparatus also allow finding a human, buried under ruins, basing on human ELF-activity.

This works in the following way. To detect the human body 90, buried under the ruins 91, the apparatus scans over the ruins 90. When the sensor 12 passes over the body 91, it registers an elevated strength of the ELF field, caused by that motion converts the elevated permanent field over the body into ELF. When detecting the area of elevated ELF, the mechanical vibrator 92 is being activated. The vibrations 93 from the vibrator 92 drive the muscles of the body 90, which respond to the drive with generating additional ELF field, which is registered by the apparatus 94.

### SUMMARY, RAMIFICATIONS AND SCOPE

Accordingly, the reader will see that the method and apparatus of this invention can be used to detect pathogenic zones, formed by two different mechanisms, and for finding humans, buried under ruins, as well. Said two mechanisms are generating ELF field by subterranean waters, and origin of the spinning electric vector due to the natural- and manmade-stimulated superposition of mutually orthogonal, phase-shifted own Earth electromagnetic fields.

Furthermore, the method and apparatus of this invention has he additional advantages in that
it permits measurement of extended set of characteristics of earth EM field: phase shift, field strength and polarization for extending received dowsing-related information in a wide spectrum band;
it provides operation of the method and instrument both in ELF and wide spectrum bands related to functioning human organs and the organism as whole.
it provides registration of spatial phenomena caused by interaction of components of EM wave, related to a landscape or other geophysical characteristics.
it makes measurement more sensitive due to eliminating unwanted sources of signal, caused both contracting muscles and moving limbs of operator;
it enlarges a field of social application of method and apparatus due to increasing sensitivity when searching human body, buried under ruins.

Although the description above contains many specificities, these should not be construed as limiting the scope of the invention but as merely providing illustrations of some of the presently preferred embodiments of this invention.

The application of The Method and The Apparatus is not limited to pathogenic zones of natural origination. It also can reveal additional inauspicious zones and situations of artificial origination. They are based on converting the Earth permanent field into the alternating field, considered above. For instance, systematically located within 3-5 m trees among the roadways make systematic distortion of earth permanent field. When driving with a speed of 30 miles/hour (13.3 m/s), crossing the line of trees creates an alternating ELF field of frequency 2.7-4.4 Hz, having the strength ˜100 V/m. This band matches that of delta- and theta-rhythms, known in the neurophysiology by their specific effects. Such a heavy field induces electric potentials under a cranium of a driver, which influence his or her ability to make decision when driving.

Another example of social-related ELF fields are those, generated by chaotically moving people due to the considered above permanent-to-alternating field conversion. This influences a social behavior of big groups of people.

The considered have to be measured and controlled with the current Method and Apparatus due to its extreme social importance.

The central frequency of the band pass filters was chosen on a base of spectral research of Ground—Ionosphere radiation. Although the embodiment of the Method shown above was operating at 6208 Hz (the center of the frequency of the band pass filters) common sense points the spinning vector phenomenon will be more profound at much higher frequencies.

There is another remark concerning the operation of the embodiment: to provide the maximum sensitivity to the phase shift the instrument should be rotate by the operator for 180°.

As it was shown above the body of the walking operator produces unwanted distortions. This is why the Method has to include the condition the interruption of the operator to provide the reading.

The prior art has disadvantages in that unwanted pulses, produced by moving limbs of operator, affect reading of the instrument. Another source of unwanted signal is generating ELF field caused by modulating earth own field by these limbs when operator walks. The great portion of the spectrum of these pulses lay in ELF region.

Beside the influence of operator's muscles, there is another source of distortions, caused by the operator. This is modulating earth own permanent electric field by moving limbs of the operator, which generates ELF electric field. To exclude this component of the distortions, reading the instrument has to be done when operator stops.

Perception of electromagnetic fields by means of conductive plates, shown in the embodiment, provides detecting electrical component of the field. Other adequate means, like mutually-orthogonal coils of inductors can be also used. In his case, the apparatus percepts magnetic component of the EM field. The combination of the conductive plates and the coils also can be used for perception of the field.

What is claimed is:

1. Method to detect pathogenic zones, said method comprising the steps of:
a. choosing a referent area with positive statistics for human health, and;
b. measuring strength of Earth electromagnetic field within the referent area in a range of electromagnetic spectrum of human organs, fixing the maximal reading, and;
c. measuring a strength of Earth electromagnetic field in different points of investigated area in the range of electromagnetic spectrum of human organs, and;
d. comparing the readings for the each point in the investigated area with that of the maximal reading for the referent area, and;
e. considering as pathogenic those points of the investigated area, for which the reading of the strength exceeds that of the maximal referent reading subject to instrument error, and;
f. mapping said readings on a plan of the investigated area;
whereby said mapping represents a distribution of pathogenic points in the investigated area.

2. The Method of claim 1, wherein the measurements are performed in the range of electromagnetic spectrum of human organs matching ELF and VLF ranges.

3. Method to detect pathogenic zones, said method comprising the step of eliciting the spinning electric and magnetic vectors, produced both by natural and artificial sources, and defining characteristics of said spinning vectors.

4. The Method of claim 3, wherein eliciting the spinning electric and magnetic vectors and defining their characteristics comprises the following steps:
a. measuring strength of electric or magnetic component of Earth alternating field for three major orthogonal vectors, which are normal to three major coordinate planes, and;
b. for each pair of the vectors, taking a vector product, and;
c. measuring a phase shift between said vectors, and;
d. taking a product of the phase shift, angular frequency of the field and said vector product and converting it into electric signal, and;
e. measuring said signals and fixing the reading, which is a value called a spinning;
f. adding the particular spinning for the major coordinate planes;
whereby the result of that is a vector, whose direction and magnitude define resulting spinning of electric or magnetic vector at chosen point.

5. Measurement apparatus to detect pathogenic zones, said apparatus comprising means for perception of electromagnetic field in three mutually-orthogonal planes, said means connected to inputs of three voltage followers, output of said followers are connected to ELF-pass filter and three band-pass filters, outputs of the ELF-pass filters are connected to an ELF-adder, while output of the ELF-adder is connected to an indicator, the outputs of said band-pass filters are connected to inputs of three phase shift meters, while outputs of the phase shift meters and the band-pass filters are connected to inputs of three vector multipliers, the output of said vector multipliers are connected to inputs of a vector adder, the output of the vector adder is connected to another output meter; whereby the indicator at the output of the ELF-adder reads the strength of electric field in ELF-range, while the meter at the output of the vector adder reads the value of the spinning.

6. Measurement apparatus according to claim 5, wherein the means for perception of electromagnetic field is three mutually-orthogonal conductive plates, while the central frequency of the band-pass filters matches the band of the Very Low Frequencies (VLF);
